(19) United States
(12) Patent Application Publication
(10) Pub. No.: US 2023/0072079 A1
(43) Pub. Date: Mar. 9, 2023

(54) Nucleic acid vaccination using neo-epitope encoding constructs

(71) Applicant: Evaxion Biotech A/S, Hørsholm (DK)

(72) Inventors: Birgitte Rønø, Hørsholm (DK); Jens Kringelum, Hørsholm (DK); Anders Bundgård Sørensen, Hørsholm (DK); Thomas Trolle, Hørsholm (DK); Stine Friis, Hørsholm (DK)

(21) Appl. No.: 17/786,631

(22) PCT Filed: Dec. 18, 2020
(86) PCT No.: PCT/EP2020/087111

§ 371 Date: Jun. 17, 2022

(30) Foreign Application Priority Data
Dec. 18, 2019 (EP) 19217713.7

(51) Int. Cl. A61K 39/00; C12N 15/117; C12N 15/85; A61K 39/39; A61P 35/00

(52) U.S. Cl. CPC A61K 39/0011; C12N 15/117; C12N 15/85; A61K 39/39; A61P 35/00; C12N 2310/17; C12N 2310/315; A61K 2039/53; A61K 2039/55561; A61K 2039/545

(57) ABSTRACT

Products and methods for DNA vaccination targeting cancer. A method for anti-cancer vaccination using a plasmid-based vaccine comprising regions encoding neo-epitopes.

### FIELD OF THE INVENTION

The present invention relates to cancer therapy, in particular cancer immunotherapy. In particular, the present invention relates to methods and products for treating cancer by nucleic acid vaccination.

### BACKGROUND OF THE INVENTION

Treatment of malignant neoplasms in patients has traditionally focused on eradication/removal of the malignant tissue via surgery, radiotherapy, and/or chemotherapy using cytotoxic drugs in dosage regimens that aim at preferential killing of malignant cells compared to killing of non-malignant cells.

In addition to the use of cytotoxic drugs, more recent approaches have focused on targeting of specific biologic markers in the cancer cells in order to reduce systemic adverse effects exerted by classical chemotherapy. Monoclonal antibody therapy targeting cancer associated antigens has proven quite effective in prolonging life expectance in a number of malignancies. While being successful drugs, monoclonal antibodies that target cancer associated antigens or antigen can by their nature only be developed to target expression products that are known and appear in a plurality of patients, meaning that the vast majority of cancer specific antigens cannot be addressed by this type of therapy, because a large number of cancer specific antigens only appear in tumours from one single patient, cf. below.

As early as in the late 1950'ies the theory of immunosurveillance proposed by Burnet and Thomas suggested that lymphocytes recognize and eliminate autologous cells—including cancer cells—that exhibit altered antigenic determinants, and it is today generally accepted that the immune system inhibits carcinogenesis to a high degree. Nevertheless, immunosurveillance is not 100% effective and it is a continuing task to device cancer therapies where the immune system's ability to eradicate cancer cells is sought improved/stimulated.

One approach has been to induce immunity against cancer-associated antigens, but even though this approach has the potential of being promising, it suffers the same drawback as antibody therapy that only a limited number of antigens can be addressed.

Many if not all tumours express mutations. These mutations potentially create new targetable antigens (neo-antigens), which are potentially useful in specific T cell immunotherapy if it is possible to identify the neo-antigens and their antigenic determinants within a clinically relevant time frame. Since it with current technology is possible to fully sequence the genome of cells and to analyse for existence of altered or new expression products, it is possible to design personalized vaccines based on neo-antigens. However, attempts at providing satisfactory clinical end-points have as today largely failed.

One mode of vaccination that has been investigated in detail since the early 1990'ies is nucleic acid vaccination (also termed DNA vaccination), where DNA is administered in a non-viral plasmid form to somatic cells of a mammal leading to expression of the genes comprised in the plasmid; in DNA vaccination the encoded material is immunogenic polypeptide(s), which upon production by the somatic cells will be able to induce an immune response. This approach is appealing as it avoids the need of producing the protein immunogen in clinical grade purity using expensive recombinant expression systems. However, it has proven difficult to obtain expression levels from the DNA administered which are high enough to effect satisfactory immune responses in humans.

There is hence an existing need for provision of anti-cancer vaccines, in particular nucleic acid vaccines, that can effectively target neo-antigens and induce clinically significant immune responses in vaccinated human beings.

### OBJECT OF THE INVENTION

It is an object of embodiments of the invention to provide methods and products for use in nucleic acid vaccination in order to treat or ameliorate cancer in larger mammals such as humans.

### SUMMARY OF THE INVENTION

It has been found by the present inventor(s) that immunization of mice with DNA vaccine plasmids encoding identified neo-epitopes of the cancer is able to provide protective immunity against the cancer.

So, in a first aspect the present invention relates to a method of inducing a therapeutic or ameliorating immune response against a malignant neoplasm in a patient, wherein the cells of the malignant neoplasm express genetic material that encode neo-epitope containing polypeptides, the method comprising administering to the patient at least one effective dosage of a composition comprising

1) at least one expression vector, which comprises nucleic acid(s) encoding at least one polypeptide, which exhibits one or more neo-epitopes of the malignant neoplasm, and

2) a pharmaceutically acceptable carrier or diluent,

whereby somatic cells in the patient are brought to express the nucleic acid(s) encoding the at least one polypeptide.

In a second aspect the invention relates to a composition comprising

1) at least one expression vector, which comprises nucleic acid(s) encoding at least one polypeptide, which exhibits one or more neo-epitopes of a malignant neoplasm, and

2) a pharmaceutically acceptable carrier or diluent,

i.e. a composition identical to the one administered in the method of the first aspect of the invention.

In third and fourth aspects, the invention relates to a composition of the second aspect 1) for use as a medicament, and 2) for use in a method of the first aspect, respectively.

### LEGENDS TO THE FIGURE

FIG. 1: Plasmid map of pVAX1.

Details are provided in the examples and SEQ ID NO: 1.

FIG. 2: Plasmid map of pVAX1 S16A.

Details are provided in the examples.

FIG. 3: Plasmid map showing preferred position of ISS in pTVG4.

FIG. 4: Graph showing tumour volumes and T-cell induction in vaccinated and mock vaccinated mice.

A: The average tumour volume measured in 13 vaccinated and 13 mock vaccinated BALB/c mice at days 0, 1, 4, 7, 9, 11, 14, 16, 18, and 21.

B: The tumour volume area under curve (AUC) measured in 13 vaccinated and 13 mock vaccinated BALB/c mice followed over the course of days 0, 1, 4, 7, 9, 11, 14, 16, 18, and 21.

C: Cytokine producing splenic CD8+ and CD4+ T cells in vaccinated and mock vaccinated mice after re-stimulation with S16A neopeptides.

D: C22 specific circulating CD8+ T cells at day 1 in vaccinated and mock vaccinated mice.

FIG. 5: Graph showing tumour volumes in vaccinated and mock vaccinated mice.

A: The average tumour volume measured in 15 vaccinated and 15 mock vaccinated BALB/c mice at days 0, 1, 4, 7, 9, 11, 14, 16, 18, and 21.

B: The tumour volume area under curve (AUC) measured in 15 vaccinated and 15 mock vaccinated BALB/c mice over the course of days 0, 1, 4, 7, 9, 11, 14, 16, 18, and 21.

C: Cytokine producing splenic CD8+ and CD4+ T cells in vaccinated and mock vaccinated mice after re-stimulation with S16A neopeptides.

D: C22 specific circulating CD8+ T cells at day 9 in vaccinated and mock vaccinated mice

FIG. 6: Graph showing tumour volumes in vaccinated and mock vaccinated mice

A: The average tumour volume measured in 14 vaccinated and 13 mock vaccinated BALB/c mice at days 0, 5, 7, 9, 12, 14, 16, and 19 following inoculation with tumour cells.

B: The tumour volume area under curve (AUC) measured in 14 vaccinated and 13 mock vaccinated BALB/c mice over the course of days 0, 5, 7, 9, 12, 14, 16, and 19.

C: C22 specific circulating CD8+ T cells at day 6 in vaccinated and mock vaccinated mice

FIG. 7: Graph showing CD8+ T cell induction in vaccinated, mock vaccinated and unvaccinated mice.

The percentages of C22 specific CD8+ T cells in tail vein blood at day 13 in mice treated with mock plasmid and two different vaccine formulations containing pVAX1 S16A (in Tyrode's buffer and PBS, respectively).

### DETAILED DISCLOSURE OF THE INVENTION

### Definitions

A “cancer specific” antigen, is an antigen, which does not appear as an expression product in an individual's non-malignant somatic cells, but which appears as an expression product in cancer cells in the individual. This is in contrast to “cancer-associated” antigens, which also appear—albeit at low abundance—in normal somatic cells, but are found in higher levels in at least some tumour cells.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

A “neo-epitope” is an antigenic determinant (typically an MHC Class I or II restricted epitope), which does not exist as an expression product from normal somatic cells in an individual due to the lack of a gene encoding the neo-epitope, but which exists as an expression product in mutated cells (such as cancer cells) in the same individual. As a consequence, a neo-epitope is from an immunological viewpoint truly non-self in spite of its autologous origin and it can therefore be characterized as a tumour specific antigen in the individual, where it constitutes an expression product. Being non-self, a neo-epitope has the potential of being able to elicit a specific adaptive immune response in the individual, where the elicited immune response is specific for antigens and cells that harbour the neo-epitope. Neo-epitopes are on the other hand specific for an individual as the chances that the same neo-epitope will be an expression product in other individuals is minimal. Several features thus contrast a neo-epitope from e.g. epitopes of tumour specific antigens: the latter will typically be found in a plurality of cancers of the same type (as they can be expression products from activated oncogenes) and/or they will be present—albeit in minor amounts—in non-malignant cells because of over-expression of the relevant gene(s) in cancer cells.

A “neo-peptide” is a peptide (i.e. a polyamino acid of up to about 50 amino acid residues), which includes within its sequence a neo-epitope as defined herein. A neo-peptide is typically “native”, i.e. the entire amino acid sequence of the neo-peptide constitutes a fragment of an expression product that can be isolated from the individual, but a neo-peptide can also be “artificial”, meaning that it is constituted by the sequence of a neo-epitope and 1 or 2 appended amino acid sequences of which at least one is not naturally associated with the neo-epitope. In the latter case the appended amino acid sequences may simply act as carriers of the neo-epitope, or may even improve the immunogenicity of the neo-epitope (e.g. by facilitating processing of the neo-peptide by antigen-presenting cells, improving biologic half-life of the neo-peptide, or modifying solubility).

A “neo-antigen” is any antigen, which comprises a neo-epitope. Typically, a neo-antigen will be constituted by a protein, but a neo-antigen can, depending on its length, also be identical to a neo-epitope or a neo-peptide.

The term “amino acid sequence” is the order in which amino acid residues, connected by peptide bonds, lie in the chain in peptides and proteins. Sequences are conventionally listed in the N to C terminal direction.

A “linker” is an amino acid sequence, which is introduced between two other amino acid sequences in order to separate them spatially. A linker may be “rigid”, meaning that it does substantially not allow the two amino acid sequences that it connects to move freely relative to each other. Likewise, a “flexible” linker allows the two sequences connected via the linker to move substantially freely relative to each other. In encoded expression products that contain more than one neo-epitope, both types of linkers are useful. However, one particular interesting linker useful in the present invention has the 12 amino acid residue sequence AEAAAKEAAAKA (SEQ ID NO: 9).

Other linkers of interest, which can be encoded by an expression vector used in the invention, are listed in the following table:

| Type | Name | Sequence |
|---|---|---|
| Flexible | FS | GSGGGA (SEQ ID NO: 3) |
| Flexible | FL | GSGGGAGSGGGA (SEQ ID NO: 4) |
| Flexible | FV1 | GSGGGAGSGGGAGSGGGA (SEQ ID NO: 5) |
| Flexible | FV2 | GSGGGAGSGGGAGSGGGAGSGGGA |
| | | (SEQ ID NO: 6) |
| Flexible | FM | GENLYFQSGG (SEQ ID NO: 7) |
| Rigid | RL1 | KPEPKPAPAPKP (SEQ ID NO: 8) |
| Rigid | RL2 | AEAAAKEAAAKA (SEQ ID NO: 9) |
| Rigid | RM | SACYCELS (SEQ ID NO: 10) |
| Flexible | | SGGGSSGGGS (SEQ ID NO: 16) |
| Flexible | | GGGGSGGGGS (SEQ ID NO: 17) |
| Flexible | | SSGGGSSGGG (SEQ ID NO: 18) |
| Flexible | | GGSGGGGSGG (SEQ ID NO: 19) |
| Flexible | | GSGSGSGSGS (SEQ ID NO: 20) |

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T-helper lymphocyte response against the combined substance constituted by the immunogen and the immunogenic carrier, and this in turn provides for improved responses against the immunogen by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

A “hapten” is a small molecule, which can neither induce or elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The term “polypeptide” is in the present context intended to mean both short peptides of from 2 to 50 amino acid residues, oligopeptides of from 50 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide (s) in a protein can be glycosylated and/or lipidated and/or comprise prosthetic groups.

### EMBODIMENTS OF THE FIRST ASPECT OF THE INVENTION

In the method disclosed herein, a therapeutic or ameliorating immune response against a malignant neoplasm is induced in a patient (preferably a human), wherein the cells of the malignant neoplasm express genetic material that encode neo-epitope containing polypeptides. The method comprises administering to the patient at least one effective dosage of a composition comprising

1) at least one expression vector, which comprises nucleic acid(s) encoding at least one polypeptide, which exhibits one or more neo-epitopes of the malignant neoplasm, and

2) a pharmaceutically acceptable carrier, diluent, or excipient, whereby somatic cells in the patient are brought to express the nucleic acid(s) encoding the at least one polypeptide.

The expression vector is typically and preferably comprised in or constituted by a plasmid, but other expression vectors can be employed. The composition of the present invention aims at ensuring delivery of “naked” DNA to cells, i.e. a DNA expression vector, which is not part of a bacterium or virus that would be able to effect introduction of the expression vector into the target cells. A vector useful in the present compositions and methods can thus be circular or linear, single-stranded or double stranded and can in addition to a plasmid also be e.g. a cosmid, mini-chromosome or episome.

Each coding (and expressible) region can be present on the same or on separate vectors; however, it is to be understood that one or more coding regions can be present on a single vector, and these coding regions can be under the control of a single or multiple promoters. This means that the expression vector can encode a separate peptide expression product for each encoded neo-epitope or that the expression vector can encode a plurality of peptide expression products, where at least some exhibit(s) several encoded neo-epitopes, of which at least some optionally are separated by peptide linkers.

In other words, in some cases only one single expression vector is administered and expressed, and this expression vector may encode a plurality of separate proteinaceous expression products or as few as 2 or even one single expression product—in this context it is only relevant whether the encoded neo-epitopes are satisfactorily presented to the immune system and the choice of their presence in separate on combined expression products is therefore of minor relevance. In preferred embodiments, the expression vector expresses at least or about 5, such as at least or about 10, at least or about 15, at least or about 20, at least or about 25, at least or about 30 proteinaceous expression products. Higher numbers are contemplated and the limit is primarily set by the number of neo-epitopes it is possible to identify from a particular neoplasm. It goes without saying that the number of encoded neo-epitopes in the expression vector(s) cannot exceed the number of neo-epitopes found in the relevant malignant tissue.

The use of peptide linkers to separate encoded neo-epitope expression products provides spatial separation between epitopes in the expression product. This can entail several advantages: linkers can ensure that each neo-epitope is presented in an optimized configuration to the immune system, but use of appropriate linkers can also minimize the problem that irrelevant immune responses are directed against “junctional epitopes” which emerge in the regions constituted by the C-terminal end of one neo-epitope and the adjacent N-terminal end of the next neo-epitope in a multi-epitope containing expression product.

Encoded peptide linkers can be either “flexible” or “rigid”, cf. the definition above, where preferred encoded linkers are set forth. Also, it is envisaged that the linker(s) used in the invention in some embodiments can be cleavable, that is, include (a) recognition site(s) for endopeptidase(s), e.g. endopeptidases such as furin, caspases, cathepsins etc.

The neo-epitopes encoded by the expression vector can be identified in a manner known per se: “deep sequencing” of the genome of the malignant cells and of the genome of healthy cells in the same individual or a standard healthy genome can identify expressed DNA sections that provide for potentially immunogenic expression products unique to the malignant cells. The identified DNA sequences can thereafter be codon-optimized (typically for expression by human cells) and included in the expression vector—either as separate expression regions of as part of larger chimeric constructs.

In order to optimize the identification and selection of the neo-epitopes that are to be expressed by the vector, any of the prediction methods available for this purpose are in practice useful. One example of a state of the art prediction algorithm is NetMHCpan-4.0 (www.cbs.dtu.dk/services/NetMHCpan-4.0/; Jurtz V et al., J Immunol (2017), ji1700893; DOI: 10.4049/jimmunol.1700893). This method is trained on a combination of classical MS derived ligands and pMHC affinity data. Another example is NetMHCstabpan-1.0 (www.cbs.dtu.dk/services/NetMHCstabpan-1.0/; Rasmussen M et al., Accepted for J of Immunol, June 2016). This method is trained on a dataset of in vitro pMHC stability measurement using an assay where each peptide is synthesized and complexed to the MHC molecule in vitro. No cell processing is involved in this assay and the environment where the pMHC stability is measured is somewhat artificial. The method is in general less accurate than NetMHCpan-4.0. U.S. Pat. No. 10,055,540 describes a method for identification of neo-epitopes using classical MS detected ligands. Other patent application publications using similar technology are WO 2019/104203, WO 2019/075112, WO 2018/195357 (MHC Class II specific), and WO 2017 106638. Finally, MHCflurry: (DOI: doi.org/10.1016/j.cels.2018.05.014; https://github.com/openvax/mhcflurry) is like NetMHCpan trained on MS detected ligand data and pMHC affinities. A peptide-MHC Class II interaction prediction method is also disclosed in a recent publication Garde C et al., Immunogenetics, DOI: doi.org/10.1007/s00251-019-01122-z. In this publication, naturally processed peptides eluted from MHC Class II are used as part of the training set and assigned the binding target value of 1 if verified as ligands and 0 if negative.

Generally, these prediction systems employ artificial neural networks (ANNs): ANNs can identify non-linear correlations: Quantification of non-linear correlations is not an easy task, since it is difficult to calculate by simple calculation. This is primarily due to non-linear correlations described with more parameters than linear correlations and probably first appear when all features are considered collectively. Hence it is needed to take all features into account in order to catch the dependency across features.

In order to further improve the likelihood that the selected encoded neo-epitopes provide for an effective immune response, use can preferably be made of the technology disclosed in European patent application Nos: 19197295.9 and 19197306.4, both filed on 13 Sep. 2019. These applications disclose technology, which enables that stability of binding between peptides and MHC molecules can be determined and which enables that stability of MHC binding of neo-epitopes is determined as part of the neo-epitope detection and selection. In brief, the data obtained from stability determinations are e.g. used as part of the training set for an ANN, and the ANN can subsequently rank identified peptides according to their predicted binding stabilities towards relevant MHC molecules.

When a nucleic acid vaccine is administered to a patient, the corresponding gene product (such as a desired antigen) is produced in the patient's body. In some embodiments, nucleic acid vaccine vectors that include optimized recombinant polynucleotides can be delivered to a human to induce a therapeutic or prophylactic immune response.

Plasmid and other naked DNA vectors are typically more efficient for gene transfer to muscle tissue. The potential to deliver DNA vectors to mucosal surfaces by oral administration has also been reported and DNA plasmids have been utilized for direct introduction of genes into other tissues than muscle. DNA vaccines have been introduced into animals primarily by intramuscular injection, by gene gun delivery, by jet injection (using a device such as a Stratis® device from PharmaJet), or by electroporation; each of these modes of administration apply to the presently disclosed method. After being introduced, the plasmids are generally maintained episomally without replication. Expression of the encoded proteins has been shown to persist for extended time periods, providing stimulation of both B and T cells.

In determining the effective amount of the vector to be administered in the treatment method disclosed herein, the physician evaluates vector toxicities, progression of the cancer to be treated, and the production of anti-vector antibodies, if any. Administration can be accomplished via single or divided doses and typically as a series of time separated administrations. In the methods disclosed herein, the effective human dose per immunization in a time-separated series is between 0.1 μg and 500 mg, with dosages between 0.1 μg and 25 mg of the expression vector being preferred. That is, in the practice of the method disclosed herein dosages of between 0.5 μg and 20 mg in humans are typically used, and dosages are normally between 5 μg and 15 mg, between 50 μg and 10 mg, and between 500 μg and 8 mg, and particular interesting dosages are of about 0.0001, about 0.0005, about 0.001, about 0.005, about 0.01, about 0.05, about 0.1, about 0.5, about 1, about 2, about 3, about 4, about 5, about 6, about 7 and about 8 mg.

A series of immunizations with effective dosages will typically constitute a series of 2, 3, 4, 5, 6, or more dosages. Multiple (e.g. >6) dosages may for instance be relevant in order to keep a malignant neoplasm in check for a prolonged period and in such a situation the exact choice of encoded neo-epitopes in the vaccine vector can be changed over time in response to changes in the genome and proteome of the malignant cells. When and if new neo-epitopes are produced by the malignant cells these can conveniently be included as targets for the vaccine.

The vaccine used in the method disclosed herein comprises one or more expression vectors; for instance, the vaccine may comprise a plurality of expression vectors each capable of autonomous expression of a nucleotide coding region in a mammalian cell to produce at least one immunogenic polypeptide. An expression vector often includes a eukaryotic promoter sequence, such as the nucleotide sequence of a strong eukaryotic promoter, operably linked to one or more coding regions. The compositions and methods herein may involve the use of any particular eukaryotic promoter, and a wide variety are known; such as a CMV or RSV promoter. The promoter can be heterologous with respect to the host cell. The promoter used may be a constitutive promoter. The promoter used may include an enhancer region and an intron region to improve expression levels, such as is the case when using a CMV promoter.

Numerous plasmids known in the art may be used for the production of nucleic acid vaccines. Suitable embodiments of the nucleic acid vaccine employ constructs using the plasmids VR1012 (Vical Inc., San Diego Calif.), pCMVI.UBF3/2 (S. Johnston, University of Texas), pTVG4 (Johnson et al., 2006, Vaccine 24(3); 293-303), pVAX1 (Thermo Fisher Scientific, see above and the Examples below), or pcDNA3.1 (InVitrogen Corporation, Carlsbad, Calif.) as the vector.

In addition, the vector construct can according to the present invention advantageously contain immunostimulatory sequences (ISS). The aim of using such sequences in the vaccine vector is to enhance T-cell response towards encoded neo-epitopes, in particular Th1 cell responses, which are elicited by adjuvants that incorporate agonists of the toll-like receptors TLR3, TLR7-TLR8, and TLR9. and/or cytosolic RNA receptors such as, but not limited to, RIG-1, MDA5 and LGP2 (Desmet et al. 2012. Nat. Rev. Imm. 12(7), 479-491)

One possibility of employing ISS is to mimic a bacterial infection activating TLR9 by stimulating with unmethylated CG-rich motifs (so-called CpG motifs) of six bases with the general sequence NNCGNN (which have a 20-fold higher frequency in bacterial DNA than in mammalian DNA) either as directly administered small synthetic DNA oligos (ODNs), which contain partially or completely phosphorothioated backbones, or by incorporating the CpG motifs in the DNA vector backbone. Immunostimulatory CpGs can be part of the DNA backbone or be concentrated in an ISS where the CpG sequence(s) typically will be positioned between the stop codon in the neo-epitope coding sequence and the poly-A tail encoding sequence (i.e. the ISS is located between the stop codon and the polyadenylation signal). However, since CpG sequences exert an effect irrespectively of their position in a longer DNA molecule, their position could in principle be anywhere in the vaccine vector as long as the presence of the CpG motif does not interfere with the vector's ability to express the coding regions of the vaccine antigen.

If present in the vaccine as separate ODNs, where the ODNs function as immunological adjuvants, CpG motif containing oligonucleotides are typically to be co-administered/formulated together with the DNA vaccine by the selected delivery technology and will typically constitute hexamers or longer multimers of DNA comprising the sequence NNCGNN or the reverse complementary sequence. Useful ODNs for this purpose are e.g. commercially available from InivoGen, 5 Rue Jean Rodier, F-31400, Toulouse, France, which markets a range of Class A, B, and C ODNs. Examples are:

ODN1585
SEQ ID NO: 21
(5′-ggGGTCAACGTTGAgggggg-3′),
ODN2216
SEQ ID NO: 22
(5′-ggGGGACGATCGTCgggggg-3′),
ODN2336
SEQ ID NO: 23
(5′-gggGACGACGTCGTGgggggg-3′),
ODN1668
SEQ ID NO: 24
(5′-tccatgacgttcctgatgct-3′),
ODN1826
SEQ ID NO: 25
(5′-tccatgacgttcctgacgtt-3′),
ODN2006
SEQ ID NO: 26
(5′-tcgtcgttttgtcgttttgtcgtt-3′),
ODN2007
SEQ ID NO: 27
(5′-tcgtcgttgtcgttttgtcgtt-3′),
ODNBW006
SEQ ID NO: 28
(5′-tcgacgttcgtcgttcgtcgttc-3′),
ODN D-SL01
SEQ ID NO: 29
(5′-tcgcgacgttcgcccgacgttcggta-3′),
ODN2395
SEQ ID NO: 30
(5′-tcgtcgttttcggcgcgcgccg-3′),
ODN M362
SEQ ID NO: 31
(5′-tcgtcgtcgttcgaacgacgttgat-3′),
ODN D-SL03
SEQ ID NO: 32
(5′-tcgcgaacgttcgccgcgttcgaacgcgg-3′),

In these 12 ODNs, upper case nucleotides are phosphodiesters, lower case nucleotides are phosphorothioates, and underlining denotes palindromic sequences.

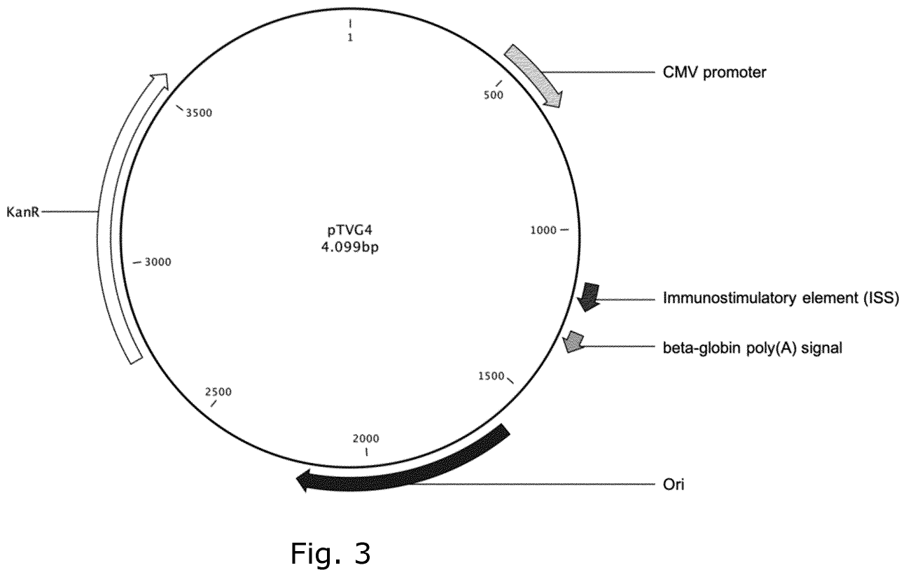

When CpG sequences are present in the plasmid backbone (which thereby become “self-adjuvating”), any number of possible NNGCNN or NNCGNN sequences can according to the invention be present, either as identical sequences or in the form of non-identical sequences of the CpG motif, or in the form of palindromic sequences that can form stem-loop structures. For instance, the following CpG motifs are of interest: AACGAC and GTCGTT, but also CTCGTT, and GCTGTT. An example of the use of such CpG encoding sequences is the following sequence excerpt from the commercially available pTVG4 vaccine vector backbone (which is schematically shown in its entirety in FIG. 3 and in SEQ ID NO: 34): . . . agatctaacgacaaaacgacaaaacgacaaggcgccagatctggcgtttcgttttgtcgttttgtcgttagatct . . . (SEQ ID NO: 33), where the underlined nucleotides constitute the CpGs that are present in the pTVG4 plasmid vector sequence.

Another possibility is to mimic an RNA viral infection to activate TLR3 by adding a double stranded(ds) RNA either as synthetic RNA oligos such as Poly I:C (polyinosinic-polycytidylic acid), poly IC:U12 (uridine substituted poly I:C), or in the form of synthetic RNA oligonucleotides (ORNs); addition of these RNA molecules to the vaccine is, as for the ODN approach, a way of obtaining an adjuvant effect. Alternatively, the dsRNA can be encoded in the DNA vector backbone, which will be transcribed into RNA after vaccination—in this case the DNA vaccine hence encodes the immunological adjuvant. This approach can include DNA sequences that encode hairpin RNA with lengths of up to 100 base pairs, where the sequence is unspecific. Also the DNA can simultaneously include ODNs and encode ORNs of known sequences; the DNA can thus both be transcribed into a double stranded RNA capable of activating TLR3 and/or cytosolic RNA receptors such as RIG-1, MDA5, and LGP2 while comprising an ODN to activate TLR9. Examples of specific DNA sequences that include/encode immune stimulating CpG and dsRNA are for instance 5′-GGTGCATCGA TGCAGGGGGG-3′ (SEQ ID NO: 35) and 5′-GGTGCATCGA TGCAGGGGGG TATATATATA TTGAGGACAG GTTAAGCTCC CCCCAGCTTA ACCTGTCCTT CAATATATA TATA-3′ (SEQ ID NO: 36) (ref: Wu et al.2011, Vaccine 29(44): 7624-30).

When ISS are present in the DNA vaccine vector, it is possible—and advantageous—to combine the approach of using CpG motifs to activate TLR9 with the presence of coding sequences for immune stimulating RNA to activate TLR3 and/or cytosolic RNA receptors such as RIG-1, MDA5, and LGP2; cf. Grossmann C et al. 2009, BMC. Immunology 10:43 and Desmet et al. 2012. Nat. Rev. Imm. 12(7), 479-491. Likewise, incorporation of ORNs and ODNs in the vaccine as separate adjuvants (alone or in combination) may be combined with the incorporation of ISS of both types in the DNA vaccine vector.

As is the case for the CpG motifs, the DNA encoding the immune stimulatory RNA ISS will preferably be present between the stop codon and the polyadenylation signal but can be present in any part of the vector as long as this does not impair the production of the intended polypeptide expression product.

In particularly important embodiments, ISS is/are comprised in the vaccine compositions, and in particular important embodiments this is achieved by incorporating an immunologically active and pharmaceutically acceptable amount of poly I:C and/or poly IC:U12. Poly I:C is constituted by a mismatched double-stranded RNA (dsRNA) with one strand being a polymer of inosinic acid and the other strand a polymer of cytidylic acid. Poly IC:U12 is a variant of poly I:C where uridine is introduced into the Poly I:C strand. These two substances will in that context function as immunological adjuvants, i.e. substances that themselves do not elicit a specific adaptive immune response, but which enhances the specific adaptive immune response against the vaccine antigen (or in the present case, the encoded antigen).

Poly I:C or poly IC:U12 (such as Ampligen®) will preferably be present in the composition so as to arrive at an administered dosage of between 0.1 and 20 mg per administration of the effective dosage of the expression vector; that is, the amount present in the composition is adjusted so as to arrive at such dosages per administration. Preferably the administered dosage of poly I:C or poly IC:U12 is between 0.2 and 15 mg per administration of the effective dosage of the expression vector, such as between 0.3 and 12, 0.4 and 10 and 0.5 and 8 mg, preferably about 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.1, 2.2, 2.3, 2.4, 2.5, 2.6, 2.7, 2.8, 2.9, 3.1, 3.2, 3.3, 3.4, 3.5, 3.6, 3.7, 3.8, 3.9 and 4.0 mg. Particularly preferred are in the range between 0.5 and 2.0 mg per administration.

The nucleic acid vaccine can also encode a fusion product containing one or more immunogenic polypeptides containing neo-epitopes. Plasmid DNA can also be delivered using attenuated bacteria as delivery system, a method that is suitable for DNA vaccines that are administered orally. Bacteria are transformed with an independently replicating plasmid, which becomes released into the host cell cytoplasm following the death of the attenuated bacterium in the host cell.

DNA vaccines, including the DNA encoding the desired antigen, can be introduced into a host cell in any suitable form including, the fragment alone, a linearized plasmid, a circular plasmid, a plasmid capable of replication, an episome, RNA, etc. Preferably, the gene is contained in a plasmid. In certain embodiments, the plasmid is an expression vector. Individual expression vectors capable of expressing the genetic material can be produced using standard recombinant techniques.

Routes of administration include, but are not limited to, intramuscular, intranasal, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. In other words, the route of administration can be selected from any one of parenteral routes, such as via the intramuscular route, the intradermal route, transdermal route, the subcutaneous route, the intravenous route, the intra-arterial route, the intrathecal route, the intramedullary route, the intrathecal route, the intraventricular route, the intraperitoneal, the intranasal route, the vaginal route, the intraocular route, or the pulmonary route; is administered via the oral route, the sublingual route, the buccal route, or the anal route; or is administered topically.

Typical routes of administration include intramuscular, intraperitoneal, intradermal and subcutaneous injection. Genetic constructs may be administered by means including, but not limited to, traditional syringes, needleless injection devices, “microprojectile bombardment gene guns”, or other physical methods such as electroporation (“EP”), “hydrodynamic method”, or ultrasound. DNA vaccines can be delivered by any method that can be used to deliver DNA as long as the DNA is expressed and the desired antigen is produced in the cell.

In some embodiments, a DNA vaccine composition disclosed herein is delivered via or in combination with known transfection reagents such as cationic liposomes, fluorocarbon emulsion, cochleate, tubules, gold particles, biodegradable microspheres, or cationic polymers. Cochleate delivery vehicles are stable phospholipid calcium precipitants consisting of phosphatidyl serine, cholesterol, and calcium; this nontoxic and noninflammatory transfection reagent can be present in a digestive system. Biodegradable microspheres comprise polymers such as poly(lactide-co-glycolide), a polyester that can be used in producing microcapsules of DNA for transfection. Lipid-based microtubes often consist of a lipid of spirally wound two layers packed with their edges joined to each other. When a tubule is used, the nucleic acid can be arranged in the central hollow part thereof for delivery and controlled release into the body of an animal.

A DNA vaccine can also be delivered to mucosal surfaces via microspheres. Bioadhesive microspheres can be prepared using different techniques and can be tailored to adhere to any mucosal tissue including those found in eye, nasal cavity, urinary tract, colon and gastrointestinal tract, offering the possibilities of localized as well as systemic controlled release of vaccines. Application of bioadhesive microspheres to specific mucosal tissues can also be used for localized vaccine action. In some embodiments, an alternative approach for mucosal vaccine delivery is the direct administration to mucosal surfaces of a plasmid DNA expression vector which encodes the gene for a specific protein antigen.

The DNA plasmid vaccines disclosed are formulated according to the mode of administration to be used. Typically, the DNA plasmid vaccines are injectable compositions, they are sterile, and/or pyrogen free and/or particulate free. In some embodiments, an isotonic formulation is preferably used. Generally, additives for isotonicity can include sodium chloride, dextrose, mannitol, sorbitol and lactose. In some embodiments, isotonic solutions such as phosphate buffered saline are preferred; one preferred solution is Tyrode's buffer. In some embodiments, stabilizers include gelatine and albumin. In some embodiments, a stabilizing agent that allows the formulation to be stable at room or ambient temperature for extended periods of time, such as LGS or other poly-cations or poly-anions is added to the formulation.

The second constituent in the composition disclosed herein is the pharmaceutically acceptable carrier, diluent, or excipient, which is preferably in the form of a buffered solution. Parenteral vehicles include sodium chloride solution, Ringer's dextrose and sodium chloride, lactated Ringer's or fixed oils. Intravenous vehicles include fluid and nutrient replenishers, electrolyte replenishers such as those based on Ringer's dextrose, and the like. Preservatives and antimicrobials include antioxidants, chelating agents, inert gases and the like. Preferred preservatives include formalin, thimerosal, neomycin, polymyxin B and amphotericin B.

In preferred embodiments, the buffered solution is the one known as “Tyrode's buffer”, and in preferred embodiments the Tyrode's buffer has the composition 140 mM NaCl, 6 mM KCl, 3 mM CaCl2), 2 mM MgCl2, 10 mM 4-(2-hydroxyethyl)-1-piperazineethanesulfonic acid (Hepes) pH 7.4, and 10 mM glucose. The concentration of the Tyrodes' buffer (or alternatives) is typically about 35% v/v, but depending on the water content of suspended plasmids, the concentration may vary considerably—since the buffer is physiologically acceptable, it can constitute any percentage of the aqueous phase of the composition. Additional carrier substances may be included and can contain proteins, sugars, etc. Such carriers may be aqueous or non-aqueous solutions, suspensions, and emulsions. Examples of non-aqueous carriers are propylene glycol, polyethylene glycol, vegetable oils such as olive oil, and injectable organic esters such as ethyl oleate. Aqueous carriers include water, alcoholic/aqueous solutions, emulsions or suspensions, including saline.

2^{nd} Aspect—Vaccine Composition

The vaccine composition, which constitutes the 2^{nd} aspect of the invention is a composition as described under the first aspect of the invention and in the claims. Hence, each and every feature and consideration that pertains to the composition, which is used in the method disclosed herein, apply mutatis mutandis to the composition of the 2^{nd} aspect of the invention.

3^{rd} and 4^{th} and Related Aspects of the Invention

The two aspects are related in that they concern the composition of the 2^{nd} aspect for a therapeutic use, i.e. the composition of the 2^{nd} aspect of the invention for use as a medicament or for use in a method according of the first aspect of the invention, respectively.

Likewise, the 4^{th} aspect of the invention also covers use of a composition of the 2^{nd} aspect in a method of the 1^{st} aspect as well as use of the components of the composition of the 2^{nd} aspect for use in the preparation of a pharmaceutical composition for use in treatment of a malignant neoplasm.

### Example 1

Experimental Vaccine Study

The objectives of the study were to test the ability a DNA vaccine of the invention to induce neo-peptide specific T cells and to monitor the impact of the vaccine on the well-being of vaccinated mice.

Plasmids for DNA vaccination were in the two first experiments based on the commercially available pVAX1™ vector available from ThermoFisher Scientific/Invitrogen. In the third experiment, the vector backbone was the pTVG4 vector, cf. above.

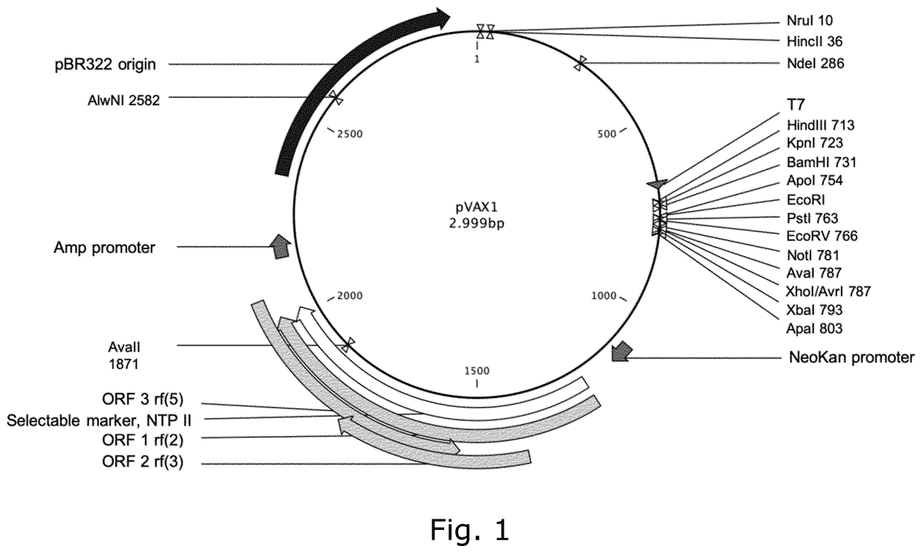

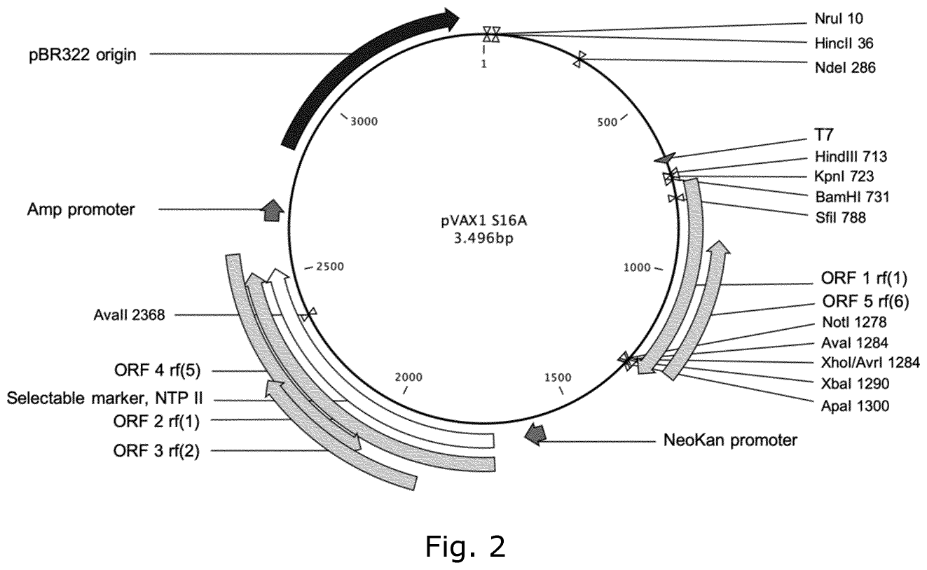

pVAX1™ is according to the manufacturer's documentation a 3.0 kb plasmid vector, which allows high-copy number replication in E. coli and high-level transient expression of encoded protein of interest in most mammalian cells. The vector (see FIG. 1) contains the following elements:

1) a human cytomegalovirus immediate-early (CMV) promoter for high-level expression in a wide range of mammalian cells,

2) a bovine growth hormone (BGH) polyadenylation signal for efficient transcription termination and polyadenylation of mRNA, and

3) A kanamycin resistance gene for selection in E. coli.

The entire sequence of the pVAX1™ plasmid is set forth in SEQ ID NO: 1. For use as a positive control for transfection and expression in the cell line of choice, one can use the control plasmid, pVAX1™/lacZ, the sequence of which is set forth in SEQ ID NO: 2.

pVAX1 S16A was constructed as follows:

Neo-peptides/neo-epitopes were first identified by whole exome sequencing of the mouse colon cancer cell line CT26 and normal tissue samples from BALB/c mice and by selecting peptides found only in the cancer cells. In the experiment, the ability of the mice to generate effective anti-tumour immune responses against the identified neo-epitopes was evaluated.

pVAX1 S16A was constructed by ligating codon-optimized (for expression in mice) DNA encoding a peptide containing the sequentially coupled 5 neo-epitopes C22, C23, C25, C30, and C38 (SEQ ID NOs: 11-15) into the pVAX1 expression cassette. The inserts also included a Kozak consensus sequence to effectively initiate translation. The encoded amino acid sequences used in the experiments are set forth in the following table:

| Peptide | AA Sequence | SEQ ID NO: |
|---|---|---|
| C22 | QIETQQRKFKASRASILSEMKMLKEKR | 11 |
| C23 | VILPQAPSGPSYATYLQPAQAQMLTPP | 12 |
| C25 | DTLSAMSNPRAMQVLLQIQQGLQTLAT | 13 |
| C30 | DGQLELLAQGALDNALSSMGALHALPR | 14 |
| C38 | RLHVVKLLASALSTNAAALTQELLVLD | 15 |

In a first experiment (EXP0233), 13 BALB/c mice were immunized i.m. in the left and right tibialis anterior muscles with plasmid pVAX1 S16A on days −13, −6, 1, 7, and 14. On day 0 the mice were inoculated with CT26 tumour cells. Also, a control group of 13 mock-vaccinated BALB/c mice were inoculated with the CT26 tumour cells. At day 21, the mice in both groups were sacrificed. Per immunization day, a total of 100 μg DNA in a mixture of 468.0 μl of H₂O and 349.1 μl of Tyrode's buffer was administered as 50 μg DNA in each thigh.

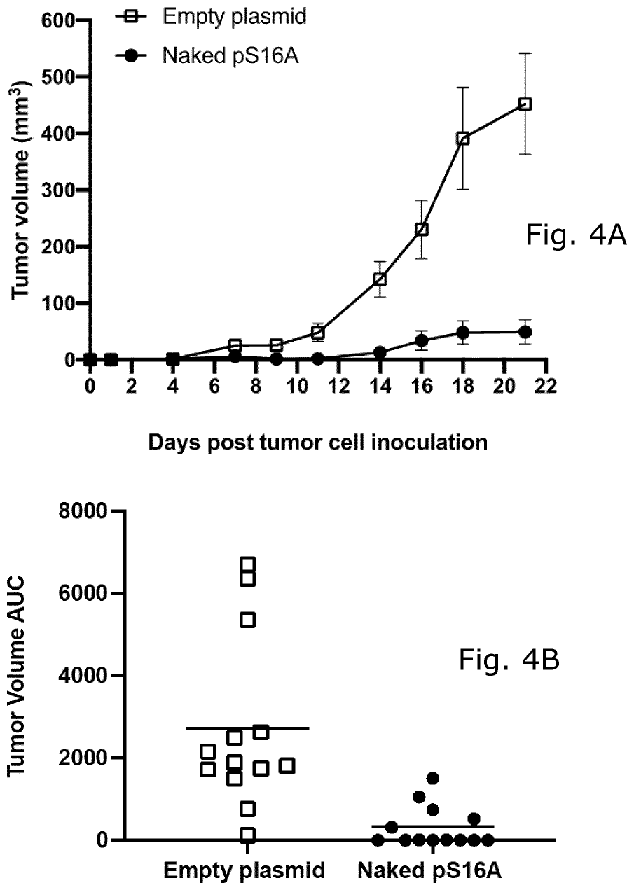

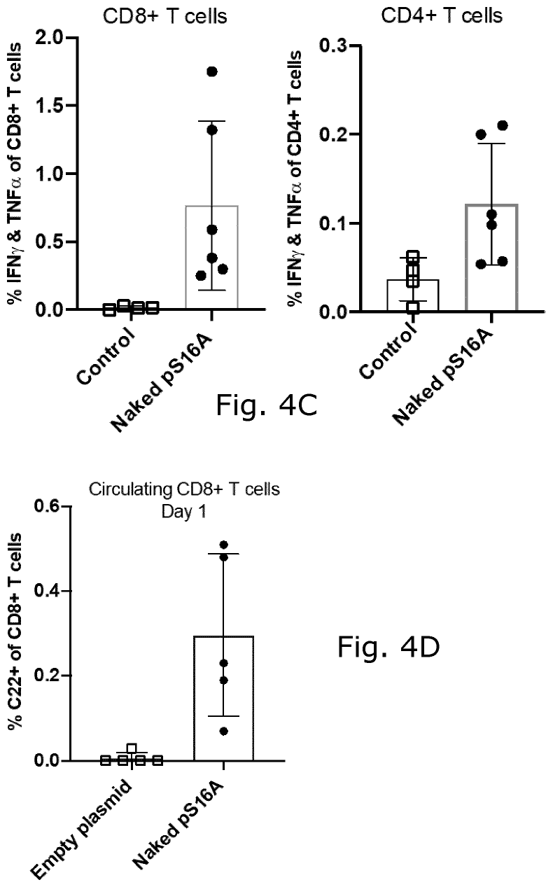

The development over time in tumour volume in the two groups of mice is shown in FIG. 4A where the average tumour volume development is shown over time. In FIG. 4B is shown the area under curve (AUC) for each test animal with an indication of the average value. The tumour growth was significantly lower in the vaccinated group than in the mock vaccinated group: For the two groups, the AUC value for the period day 0-day 21 in the neoepitope-vaccinated and mock vaccinated groups were 322 (SEM=138) and 2712 (SEM=577), respectively.

Further, the T-cell induction of the vaccine was investigated by gauging splenic T cell activation upon peptide re-stimulation. Splenic cells were re-stimulated with a pool of 27mer neo-peptides (C22, C23, C25, C30, C38) corresponding to the neo-epitope content of the S16A pentatope: It turned out that immunization with 100 μg of pVAX1 S16A Neovector DNA induced S16A neoepitope reactive splenic CD8+ T cells (FIG. 4C, let panel), seen as cytokine producers upon S16A neopeptide stimulation and ICS, whereas there was no or low cytokine signal in the samples from mock treated controls. Immunization with 100 μg of S16A Neovector DNA also induced S16A neoepitope reactive splenic CD4+ T cells (FIG. 4C, right panel), seen as cytokine producers upon S16A neopeptide stimulation and ICS. Low or no cytokine signal was detected in control samples from the mock treated controls. CD8+ T cells specific to CT26 neo-peptide C22 (H-2Kd minimal binder KFKASRASI) were observed in tail vein blood from study day 1 (one day after the third immunization) in mice immunized with naked S16A Neovector (FIG. 4D).

In a similar second experiment (EXP0264), the anti-tumour effect of pVAX1 S16A was tested in two groups (n=15) of BALB/c mice that were treated with pVAX1 S16A naked plasmid and a Kolliphor® P188 solution without plasmid. Vaccine compositions, dosages, route of administration, inoculation with CT26 and sacrifice of animals were the same as in the first experiment.

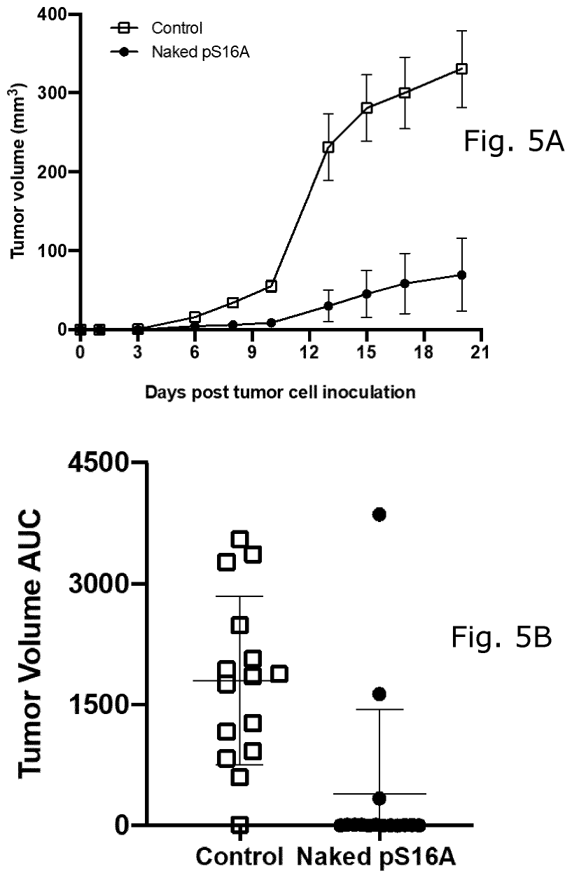

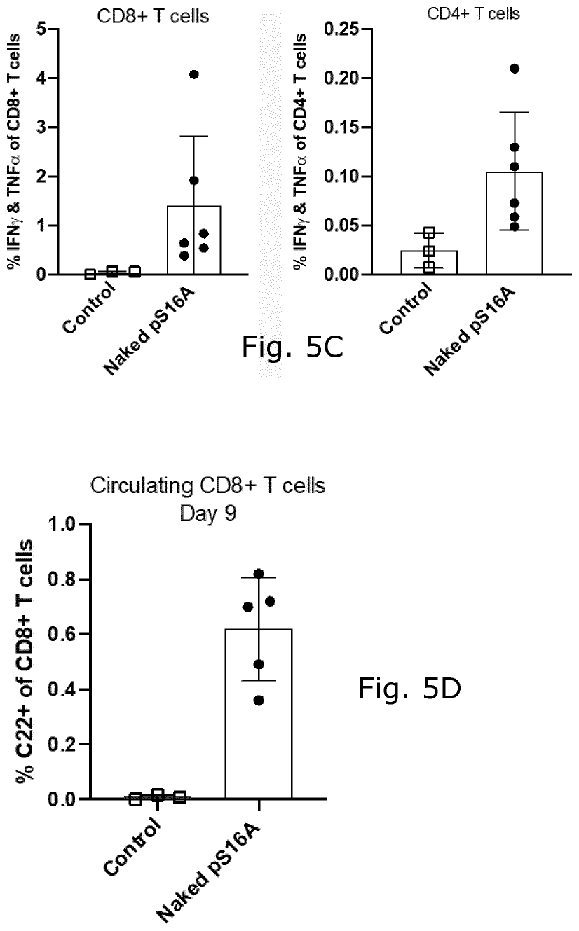

The effect on the growth of tumour cells (measured as volume of tumours) is shown in FIGS. 5A and 5B (showing the average tumour sizes over time and the tumour volume AUC, respectively, in each group of 15 mice). Expressed as AUC in the two groups, the pVAX1 16A treated mice exhibited a significantly lower growth than mock vaccinated mice: mean AUC of 394 (SEM=102) for the vaccinated group, whereas the mock plasmid treated group exhibited a mean AUC of 1702 (SEM=439) and a median AUC of 1427.

The analysis for T-cell induction (performed as in experiment 1) revealed that immunizations with 100 μg pVAS1 S16A Neovector induced CD8+ T cells, which produce cytokines IFNγ and TNFα upon S16A peptide pool stimulation (FIG. 5C, left panel). Immunizations with 100 μg S16A Neovector induced similar levels of CD4+ T cells that produce cytokines IFNγ and TNFα above background upon S16A peptide pool stimulation (FIG. 5C, right panel).

Additionally, it was found that CD8+ T cells specific to CT26 neo-peptide C22 were observed in tail vein blood at study day 9 in mice immunized with pVAX1 S16A Neovector. There were no observable C22-specific CD8+ T cells in tail vein blood from control immunized mice, cf. FIG. 5D.

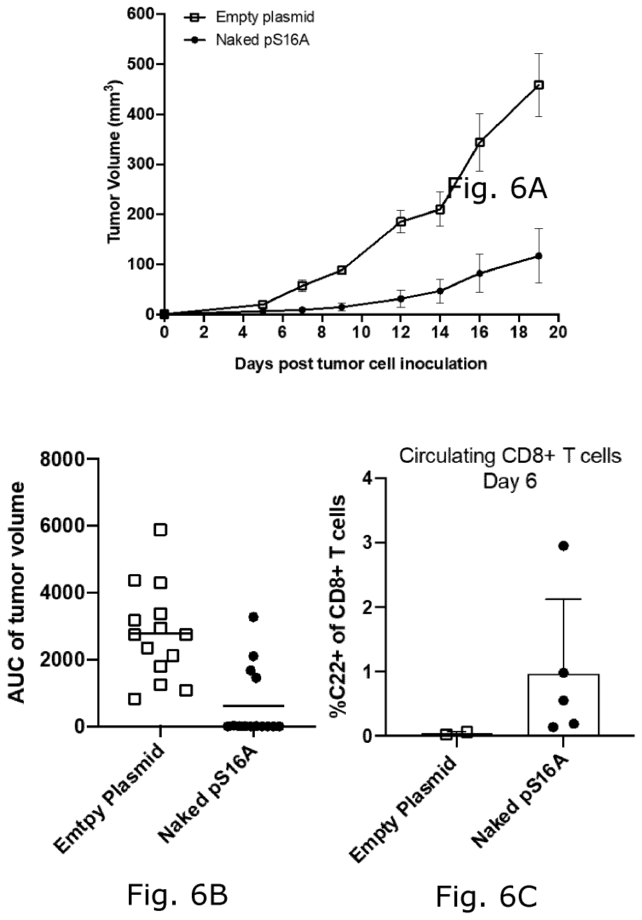

In a third experiment (EXP0287), the S16A pentatope encoding sequence was introduced in the pVTG4 vector (cf. above) and tested for anti-tumour activity in a setting as in the first experiment: 14 BALB/c mice were immunized on days on days −14, −7, 1, 7, and 14 relative to inoculation with the CT26 cells and tumour volumes were recorded after the inoculation. As is apparent from FIG. 6A and FIG. 6B, also this vector format provides for a significant inhibition of tumour growth when compared to the untreated controls from the first experiment and as observed in the 2 first experiments, circulating CD8+ T cells isolated from tail vein blood were found on day 9 in the treated group of mice.

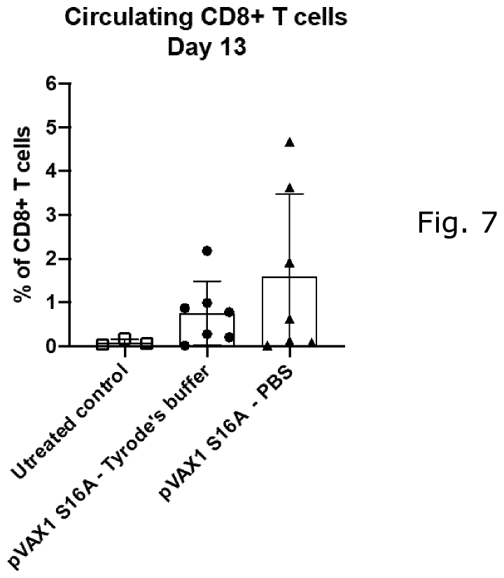

Finally, in a 4th experiment (EXP0372), groups of mice were immunized with pVAX1 S16A formulated in PBS or Tyrode's buffer or immunized with a mock plasmid. Immunizations were administered on days 0, 7, 15, 21, and 28. Tail vein blood was collected at days 5, 13 and 20. As shown in FIG. 7, the vaccinated mice receiving both formulations of pVAX1 S16A exhibited C22 specific CD8+ T cells in circulation on day 13 (i.e. after 2 immunizations), whereas the mock immunized animals did not.

1. A method of inducing a therapeutic or ameliorating immune response against a malignant neoplasm in a patient, wherein the cells of the malignant neoplasm express genetic material that encode neo-epitope containing polypeptides, the method comprising administering to the patient at least one effective dosage of a composition comprising
1) at least one DNA expression vector, which comprises nucleic acid(s) encoding at least one polypeptide, which exhibits one or more neo-epitopes of the malignant neoplasm, and
2) a pharmaceutically acceptable carrier, diluent, or excipient,
whereby somatic cells in the patient are brought to express the nucleic acid(s) encoding the at least one polypeptide,
wherein the DNA expression vector comprises or encodes at least one Immune Stimulatory Sequence (ISS).

2. The method according to claim 1, wherein the pharmaceutically acceptable carrier, diluent, or excipient is an aqueous buffered solution.

3. The method according to claim 2, wherein the aqueous buffered solution is Tyrode's buffer.

4. The method according to claim 3, wherein the Tyrode's buffer has the composition 140 mM NaCl, 6 mM KCl, 3 mM CaCl₂, 2 mM MgCl₂, 10 mM 4-(2-hydroxyethyl)-1-piperazineethanesulfonic acid (Hepes) pH 7.4, and 10 mM glucose.

5. The method according to claim 1, wherein the concentration of Tyrodes' buffer is about 35% v/v.

6. The method according to claim 1, wherein the composition further comprises at least one immune stimulating sequence (ISS).

7. The method according to claim 6, wherein the ISS is an oligodeoxyribonucleotide (ODN) comprising at least one CpG motif, and wherein the ODN preferably includes phosphorothioate groups.

8. The method according to claim 6, wherein the ISS is or comprises an oligoribonucleotide.

9. The method according to claim 8, which comprises an immunologically active and pharmaceutically acceptable amount of a stimulant of Toll-like receptor 3 (TLR-3) and/or MDA5 and/or RIG-1, such as poly I:C and/or poly IC:U12.

10. The method according to claim 9, wherein poly I:C or poly I:CU12 is present in the composition so as to arrive at an administered dosage of between 0.1 and 20 mg per administration of the effective dosage of the expression vector.

11. The method according to claim 10, wherein the administered dosage of poly I:C or poly I:CU12 is between 0.2 and 15 mg per administration of the effective dosage of the expression vector, such as between 0.3 and 12, 0.4 and 10 and 0.5 and 8 mg, preferably about 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.1, 2.2, 2.3, 2.4, 2.5, 2.6, 2.7, 2.8, 2.9, 3.1, 3.2, 3.3, 3.4, 3.5, 3.6, 3.7, 3.8, 3.9, and 4.0 mg.

12. The method according to claim 1, wherein the effective dosage contains between 0.1 μg and 25 mg of the expression vector, such as between 0.5 μg and 20 mg, between 5 μg and 15 mg, between 50 μg and 10 mg, and between 500 μg and 8 mg, in particular about 0.0001, about 0.0005, about 0.001, about 0.005, about 0.01, about 0.05, about 0.1, about 0.5, about 1, about 2, about 3, about 4, about 5, about 6, about 7 and about 8 mg.

13. The method according to claim 1, wherein the expression vector expresses at least or about 5, such as at least or about 10, at least or about 15, at least or about 20, at least or about 25, and at least or about 30 neo-epitopes.

14. The method according to claim 1, wherein the expression vector encodes a separate peptide for each encoded neo-epitope.

15. The method according to claim 1, wherein the expression vector encodes a plurality of peptides, where at least one exhibit(s) several encoded neo-epitopes, of which at least some optionally are separated by peptide linkers.

16. (canceled)

17. The method according to claim 1, wherein the at least one ISS comprised in the expression vector or ISS encoding sequence of the expression vector is/are positioned between the stop codon of a neo-epitope encoding sequence and a polyadenylation signal.

18. The method according to claim 1, which comprises at least one ISS in the expression vector.

19. The method according to claim 1, wherein at least one ISS encoding sequence is encoded by the expression vector.

20. The method according to claim 18, wherein the ISS is or comprises a sequence that activates Toll-like receptor 9 (TLR-9), such as a CpG motif.

21. The method according to claim 19, wherein the ISS encodes an RNA sequence that activates Toll-like receptor 3 (TLR-3) and/or cytosolic RNA receptors such as RIG-1, MDA5, and LGP2, such as an RNA sequence the forms an RNA hairpin or constitute an immune stimulating RNA sequence.

22. The method according to claim 1, wherein the expression vector is comprised in or constitutes a plasmid.

23. The method according to claim 1, wherein the at least one effective dosage is a series of dosages, such as a series of 2, 3, 4, 5, 6, or more dosages.

24. The method according to claim 1, wherein the patient is a human being.

25. The method according to claim 1, wherein the effective dose is administered parenterally, such as via the intramuscular route, the intradermal route, transdermal route, the subcutaneous route, the intravenous route, the intra-arterial route, the intratechal route, the intramedullary route, the intrathecal route, the intraventricular route, the intraperitoneal, the intranasal route, the vaginal route, the intraocular route, or the pulmonary route; is administered via the oral route, the sublingual route, the buccal route, or the anal route; or is administered topically.

26. (canceled)

27. (canceled)

28. (canceled)

29. (canceled)


SEQUENCE LISTING (36 sequences)

SEQ ID NO: 1 - DNA, 2999 bp, Artificial sequence
  pVAX1 DNA vaccine vector
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 2 - DNA, 6050 bp, Artificial sequence
  pVAX1/LacZ DNA vaccine vector
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatcgaaacgatgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggtaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 3 - protein, 6 aa, Artificial sequence
  Flexible linker peptide
GSGGGA

SEQ ID NO: 4 - protein, 12 aa, artificial sequence
  Flexible linker peptide
GSGGGAGSGGGA

SEQ ID NO: 5 - protein, 18 aa, Artificial sequence
  Flexible linker peptide
GSGGGAGSGGGAGSGGGA

SEQ ID NO: 6 - protein, 24 aa, Artificial sequence
  Flexible linker peptide
GSGGGAGSGGGAGSGGGAGSGGGA

SEQ ID NO: 7 - protein, 10 aa, Artificial sequence
  Flexible linker peptide
GENLYFQSGG

SEQ ID NO: 8 - protein, 12 aa, Artificial sequence
  Rigid linker peptide
KPEPKPAPAPKP

SEQ ID NO: 9 - protein, 12 aa, Artificial sequence
  Rigid linker peptide
AEAAAKEAAAKA

SEQ ID NO: 10 - protein, 8 aa, Artificial sequence
  Rigid linker peptide
SACYCELS

SEQ ID NO: 11 - protein, 27 aa, Artificial sequence
  Neo-epitope
QIETQQRKFKASRASILSEMKMLKEKR

SEQ ID NO: 12 - protein, 27 aa, Artificial sequence
  Neo-epitope
VILPQAPSGPSYATYLQPAQAQMLTPP

SEQ ID NO: 13 - protein, 27 aa, Artificial sequence
  Neo-epitope
DTLSAMSNPRAMQVLLQIQQGLQTLAT

SEQ ID NO: 14 - protein, 27 aa, Artificial sequene
  Neo-epitope
DGQLELLAQGALDNALSSMGALHALPR

SEQ ID NO: 15 - protein, 27 aa, Artificial sequence
  Neo-epitope
RLHVVKLLASALSTNAAALTQELLVLD

SEQ ID NO: 16 - protein, 10 aa, Artificial sequence
  Peptide linker
SGGGSSGGGS

SEQ ID NO: 17 - protein, 10 aa, Artificial sequence
  Peptide linker
GGGGSGGGGS

SEQ ID NO: 18 - protein, 10 aa, Artificial sequence
  Peptide linker
SSGGGSSGGG

SEQ ID NO: 19 - protein, 10 aa, Artificial sequence
  Peptide linker
GGSGGGGSGG

SEQ ID NO: 20 - protein, 10 aa, Artificial seuence
  Peptide linker
GSGSGSGSGS

SEQ ID NO: 21 - DNA, 20 bp, Artificial sequence
  CpG motif
ggggtcaacgttgagggggg

SEQ ID NO: 22 - DNA, 20 bp, Artificial sequence
  CpG motif
gggggacgatcgtcgggggg

SEQ ID NO: 23 - DNA, 21 bp, Artificial sequence
  CpG motif
ggggacgacgtcgtggggggg

SEQ ID NO: 24 - DNA, 20 bp, Artificial sequence
  CpG motif
tccatgacgttcctgatgct

SEQ ID NO: 25 - DNA, 20 bp, Artificial sequence
  CpG motif
tccatgacgttcctgacgtt

SEQ ID NO: 26 - DNA, 24 bp, Artificial sequence
  CpG motif
tcgtcgttttgtcgttttgtcgtt

SEQ ID NO: 27 - DNA, 22 bp, Artificial sequence
  CpG motif
tcgtcgttgtcgttttgtcgtt

SEQ ID NO: 28 - DNA, 23 bp, Artificial sequence
  CpG motif
tcgacgttcgtcgttcgtcgttc

SEQ ID NO: 29 - DNA, 26 bp, Artificial sequence
  CpG motif
tcgcgacgttcgcccgacgttcggta

SEQ ID NO: 30 - DNA, 22 bp, Artificial sequence
  CpG motif
tcgtcgttttcggcgcgcgccg

SEQ ID NO: 31 - DNA, 25 bp, Artificial sequence
  CpG motif
tcgtcgtcgttcgaacgacgttgat

SEQ ID NO: 32 - DNA, 29 bp, Artificial sequence
  CpG motif
tcgcgaacgttcgccgcgttcgaacgcgg

SEQ ID NO: 33 - DNA, 75 bp, Artificial sequence
  Fragment of pTVG4 vaccine vector
agatctaacgacaaaacgacaaaacgacaaggcgccagatctggcgtttcgttttgtcgttttgtcgttagatct

SEQ ID NO: 34 - DNA, 4099 bp, Artificial sequence
  pTVG4 vaccine vector
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcacgtgggcccggtaccgtatactctagagcggccgcggatccagatctaacgacaaaacgacaaaacgacaaggcgccagatctggcgtttcgttttgtcgttttgtcgttagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 35 - DNA, 20 bp, Artificial sequence
  CPG encoding sequence
ggtgcatcgatgcagggggg

SEQ ID NO: 36 - DNA, 83 bp, Artificial sequence
  CPG encoding sequence
ggtgcatcgatgcaggggggtatatatatattgaggacaggttaagctccccccagcttaacctgtccttcaatatatatata